(19) United States
(12) Patent Application Publication
(10) Pub. No.: US 2010/0168432 A1
(43) Pub. Date: Jul. 1, 2010

(54) Process for the Preparation of Montelukast

(75) Inventors: Chava Satyanarayana, Secunderabad (IN); Gorantla Seeta Ramanjaneyulu, Secunderabad (IN); Indukuri Venkata Sunil Kumar, Secunderabad (IN)

Correspondence Address: SCHWEITZER CORNMAN GROSS & BONDELL LLP, 292 MADISON AVENUE - 19th FLOOR, NEW YORK, NY, 10017, US

(73) Assignee: MATRIX LABORATORIES LTD, Secunderabad (IN)

(21) Appl. No.: 12/083,487

(22) PCT Filed: Jun. 30, 2006
(86) PCT No.: PCT/IN2006/000226

§ 371 Date: Apr. 14, 2008

(30) Foreign Application Priority Data
Jul. 5, 2005 (IN) 872/CHE/2005

(51) Int. Cl. C07D 215/14

(52) U.S. Cl. 546/174

(57) ABSTRACT

The present invention relates to a process for the preparation of Montelukast or a salt thereof comprises, i) condensing Methyl-2-[3-[3-(2-(7-chloroquinolin-2-yl)ethenyl]phenyl]-3-chloro propyljbenzoate obtained above with 1-(mercaptomethyl)cyclopropane acetic acid in presence of alkali carbonates such as Cesium Carbonate, followed by salification with organic amine ii) neutralizing the organic amine salt as obtained above with organic/inorganic acid followed by reaction with methylmagnesium chloride in the presence of Cerium Chloride followed by treatment with organic base yields montelukast amine salt iii) converting the montelukast amine salt to Montelukast Free acid iv) saltification of Montelukast free acid with Sodium hydroxide affords Montelukast Sodium.

The present invention relates to a process for the preparation of Montelukast or its pharmaceutically acceptable salts.

### BACKGROUND OF THE INVENTION

Montelukast sodium namely Sodium salt of 1-[[[(1R)-1-[3-[(1E)-2-(7-chloro-2-quinolinyl)ethenyl]phenyl]-3-[2-(1 hydroxy-1-methylethyl)phenyl]propyl]thio]methyl]cyclopropaneacetic acid has the formula

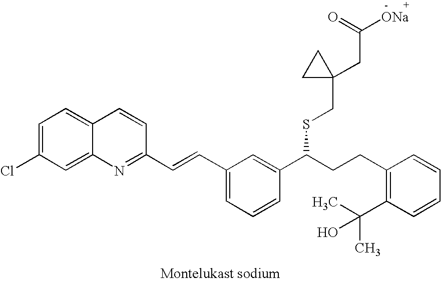

Montelukast sodium is a leukotriene antagonist and inhibits the synthesis of leukotriene biosynthesis. It is useful as anti-asthmatic, anti-allergic, anti-inflammatory, cytoprotective agent and hence useful in the treatment of angina, cerebral spasm, glomerular nephritis, hepatic, and toxemia, and uveitis and allograft rejection.

EP Pat. No. 480,717 discloses Montelukast sodium along with other related compounds and the methods for their preparation. The reported method of synthesis proceeds through corresponding methyl ester namely, Methyl 2-[(3S)-[3-[(2E)-(7-chloroquinolin-2yl)ethenyl]phenyl]-3-hydroxypropyl]benzoate and involves coupling methyl 1-(mercaptomethyl)cyclopropaneacetate with a mesylate generated in-situ. The methyl ester of Montelukast is hydrolyzed to free acids and the latter converted directly to Montelukast Sodium salt (Scheme-1). The process is not particularly suitable for large-scale production because it requires tedious chromatographic purification of the methyl ester intermediate and/or the final product and the product yield is low.

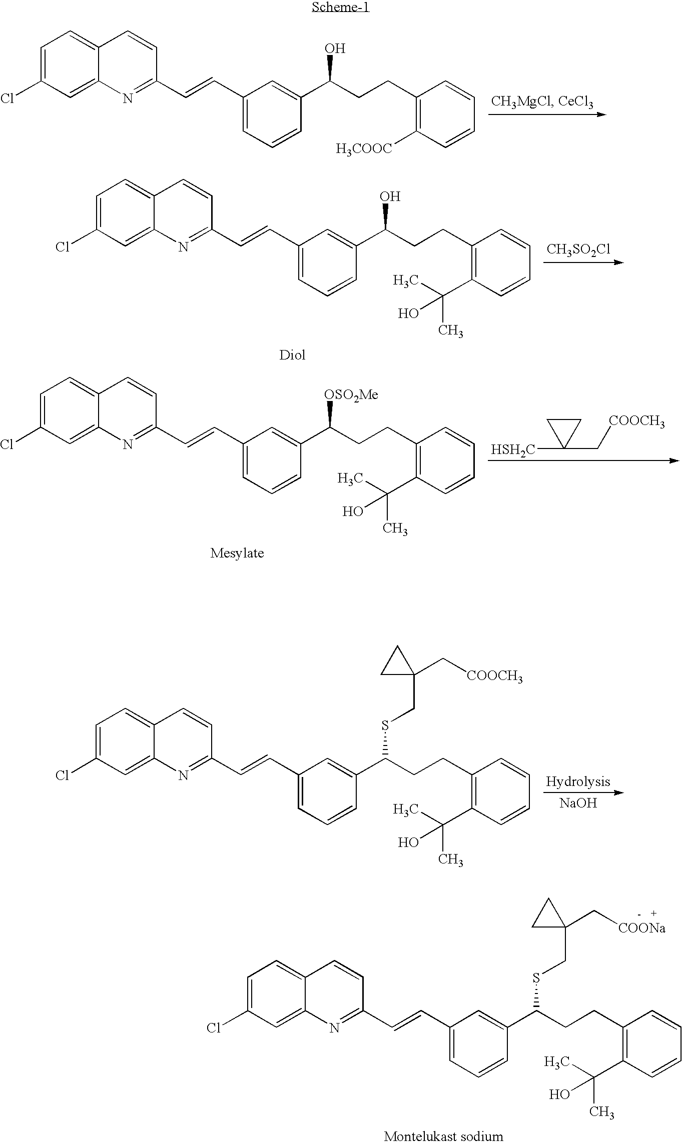

U.S. Pat. No. 5,614,632 disclosed a process for the preparation of crystalline Montelukast sodium, which comprises of the following steps (Scheme-2):
Reaction of methyl 2-[3(S)-[3-[2-(7-chloroquinolin-2-yl)ethenyl]phenyl]-3-hydroxypropyl benzoate (I) with Grignard reagent, methyl magnesium chloride in presence of cerium chloride to give Diol (II)
Reaction of Diol (II) with methane sulfonyl chloride to afford 2-[2-[3(s)-[3-(2-(7-chloro quinolin-2yl)ethenyl]phenyl]-3-methane sulfonyloxy propyl]phenyl]-2-propanol (III)
Condensation of 2-[2-[3(s)-[3-(2-(7-chloro quinolin-2-yl)ethenyl]phenyl]-3-methane sulfonyloxypropyl]phenyl]-2-propanol (III) with dilithium anion of 1-mercaptomethyl)cyclopropaneacetic acid, which has been generated by the reaction of 1-(mercaptomethyl)cyclopropaneacetic acid (IV) with n-Butyl lithium
Isolation of the condensed product, Montelukast as solid Montelukast dicyclohexylamine salt
Purification and conversion of Montelukast dicyclohexylamine salt into Montelukast sodium
Crystallization of Montelukast sodium from a mixture of toluene and acetonitrile

The process disclosed in U.S. Pat. No. 5,614,632 further involved the reaction of Diol (II) with methane sulfonyl chloride involves the reaction temperature of about −25° C. and the storage condition of the intermediate, 2-[2-[3(s)-[3-(2-(7-chloro quinolin-2-yl)ethenyl]phenyl-3-methane sulfonyloxy propyl]phenyl]-2-propanol (III) at temperature below −15° C. for having the stability. The process further involves the reaction, formation of dilithium anion of 1-(mercaptomethyl)cyclopropaneacetic acid which requires the usage of n-Butyl lithium, a highly flammable and hazardous reagent and the reaction is at temperature below −5° C. further requires anhydrous conditions.

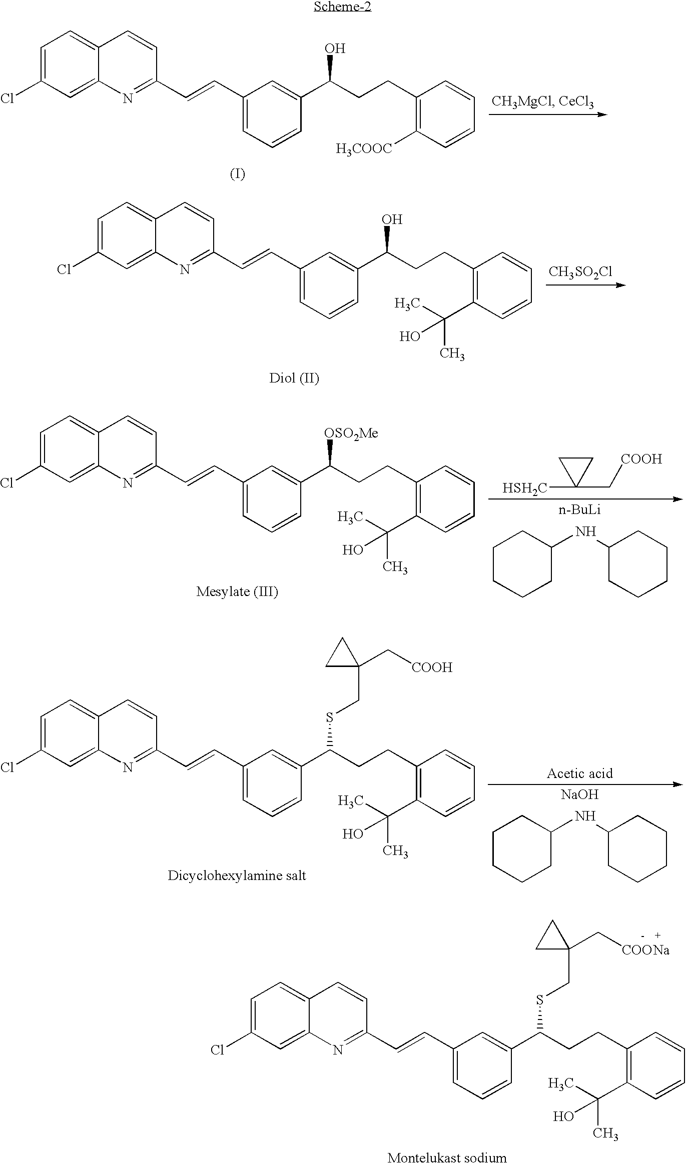

In our co pending PCT application PCT/IN04/00211 Montelukast is being prepared by
Reacting Methyl 2-[(3S)-[3-[(2E)-(7-chloro quinolin-2yi)ethenyl]phenyl]-3-halopropyl]benzoate with 1-(mercaptomethyl)cyclopropane acetic acid in presence of alkali hydride or alkoxide
Isolating the product 2-[1-[1(R)[3-[2-(7-chloroquinolin-2yl)ethenyl]phenyl]-3-[2-(methoxycarbonyl)phenyl]propysulfanylmethyl]cyclopropane]acetic acid as organic amine salt.
Neutralizing the 2-[1-[1(R)-[3-[2-(7-chloroquinolin-2-yl)ethenyl]phenyl]-3-[2-(methoxycarbonyl)phenyl]propyl]propyl sulfanylmethyl cyclopropane acetic acid organic amine salt followed by reaction with Grignard reagent to give Montelukast
Isolating the Montelukast as crystalline free acid or optionally as Montelukast organic base salt by reaction with organic amines

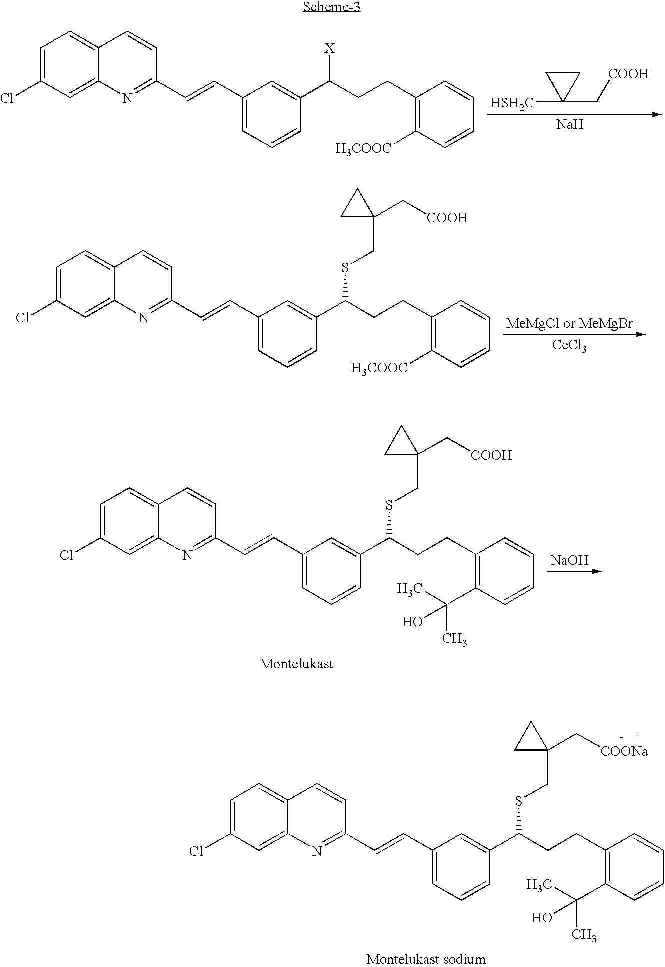

The disclosed process involves the reaction of Methyl 2-[(3S)-[3-[(2E)-(7-chloroquinolin-2-yl)ethenyl]phenyl]-3-halopropyl]benzoate with 1-(mercapto methyl)cyclopropane acetic acid in presence of alkali hydride or alkoxide. The alkali hydrides are strong bases, hazardous in nature, Reacts violently with water liberates hydrogen gas and special precautions to be taken for handling.

### SUMMARY OF THE INVENTION

The main object of the present invention is to provide an alternate process for the preparation of Montelukast, without involving the unstable or limited stable intermediates and further to Montelukast alkali slats.

Another object of the invention is to provide a process for the preparation of Montelukast and its alkali salts without involving the low temperature (−25° C.) reactions and storage conditions at lower temperature (−15° C.).

Another object of the invention is to provide a process for the preparation of Montelukast and its alkali salts without involving the hazardous chemicals i.e. Sodium hydride

A further aspect of the present invention relates to a process, which comprises preparing montelukast that includes.
i) Condensing Methyl-2[3-[3-(2-(7-chloroquinolin-2-yl)ethenyl]phenyl]-3-chloropropyl]benzoate with 1-(mercaptomethyl)cyclopropane acetic acid and isolating 2-[1-[1(R)-[3-[2[(7-Chloroquinolin-2-yl)ethenyl]phenyl]-3-[2-(methoxy carbonyl)phenyl]propyl sulfanyl methyl]cyclopropane) acetic acid as its organic amine salt.
ii) Neutralizing the amine salt followed by reaction with methyl magnesium chloride in the presence of anhydrous Cerium chloride and isolating the crude montelukast to its organic amine salt such as (R)-α-methyl benzyl amine salt and cyclohexyl ethylamine salt
iii) Neutralizing the montelukast organic amine salts, separating and isolating the montelukast
iv) Converting montelukast free acid to its sodium salt

### BRIEF DESCRIPTION OF THE DRAWINGS

FIG. 1: FTIR spectrum of 2-[1-[1(R)-[3-[2-(7-Chloroquinolin-2-yl)ethenyl)phenyl]-3-[2-(methoxy carbonyl)phenyl]propyl sulfanyl methyl]cyclopropyl]acetic acid α-methyl benzyl amine salt

FIG. 2: FTIR spectrum of 2-[1-[1(R)-[3-[2-(7-Chloroquinolin-2-yl)ethenyl)phenyl]-3-[2-(methoxycarbonyl)phenyl]propyl sulfanyl methyl]cyclopropyl]acetic acid (S)-cyclohexyl ethyl amine salt

FIG. 3: FTIR spectrum of 2-[1-[1(R)-[3-[2-7-Chloroquinolin-2-yl)ethenyl]phenyl]-3-[2-(1-hydroxy-1-methylethyl)phenyl]propyl sulfanyl methyl]cyclopropyl]acetic acid (S)-cyclohexyl ethyl amine salt

### DETAILED DESCRIPTION OF THE INVENTION

The process of the patent invention for the preparation of Montelukast and its salts comprises of:
Condensing Methyl-2-[3-[3-(2-(7-chloroquinolin-2-yl)ethenyl]phenyl]-3-chloro propyl]benzoate with 1-(mercaptomethyl)cyclopropane acetic acid in presence of alkali carbonates such as Cesium Carbonate, followed by saltification with organic amine
Neutralizing the organic amine salt as obtained above with organic/inorganic acid followed by reaction with methylmagnesium chloride in the presence of anhydrous Cerium Chloride followed by treatment with organic base yields montelukast amine salt
Converting the montelukast amine salt to Montelukast Free acid
Saltification of Montelukast free acid with Sodium hydroxide affords Montelukast sodium.

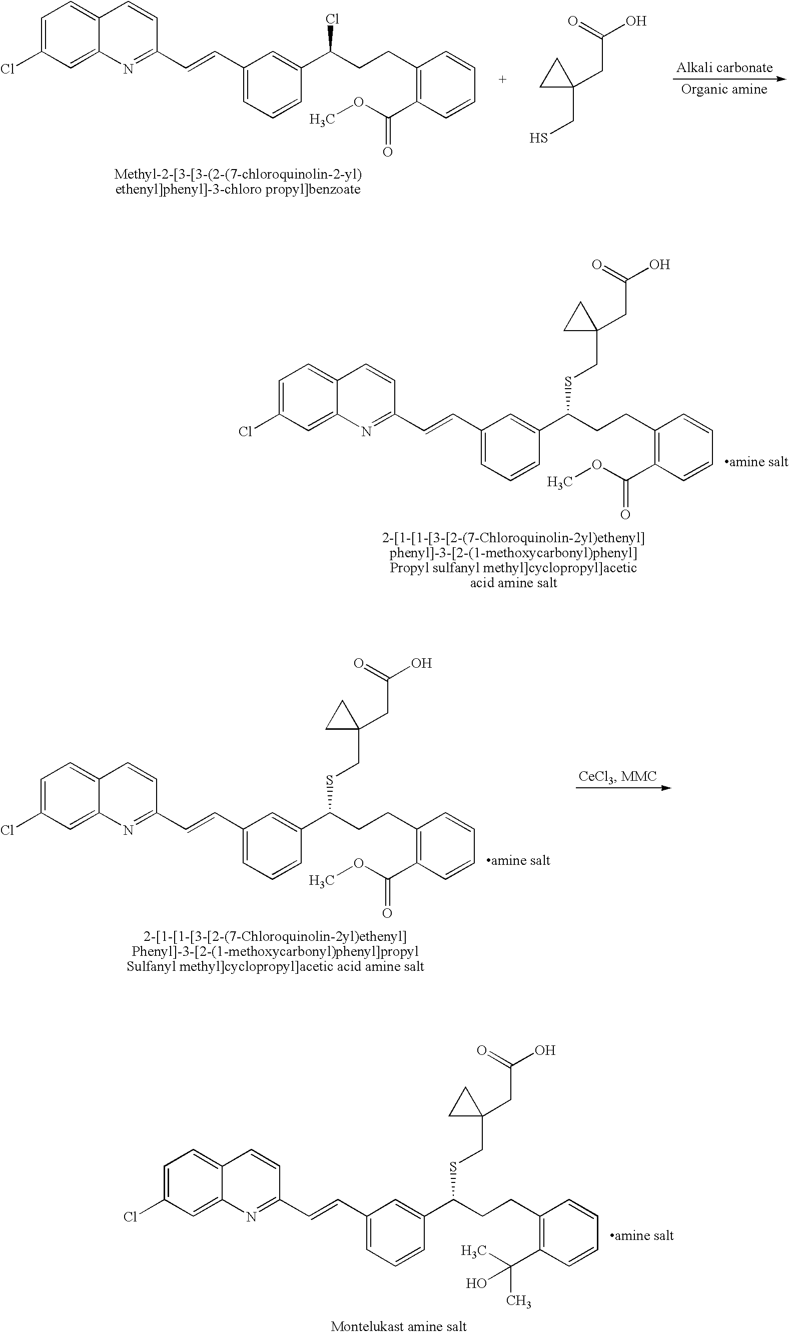

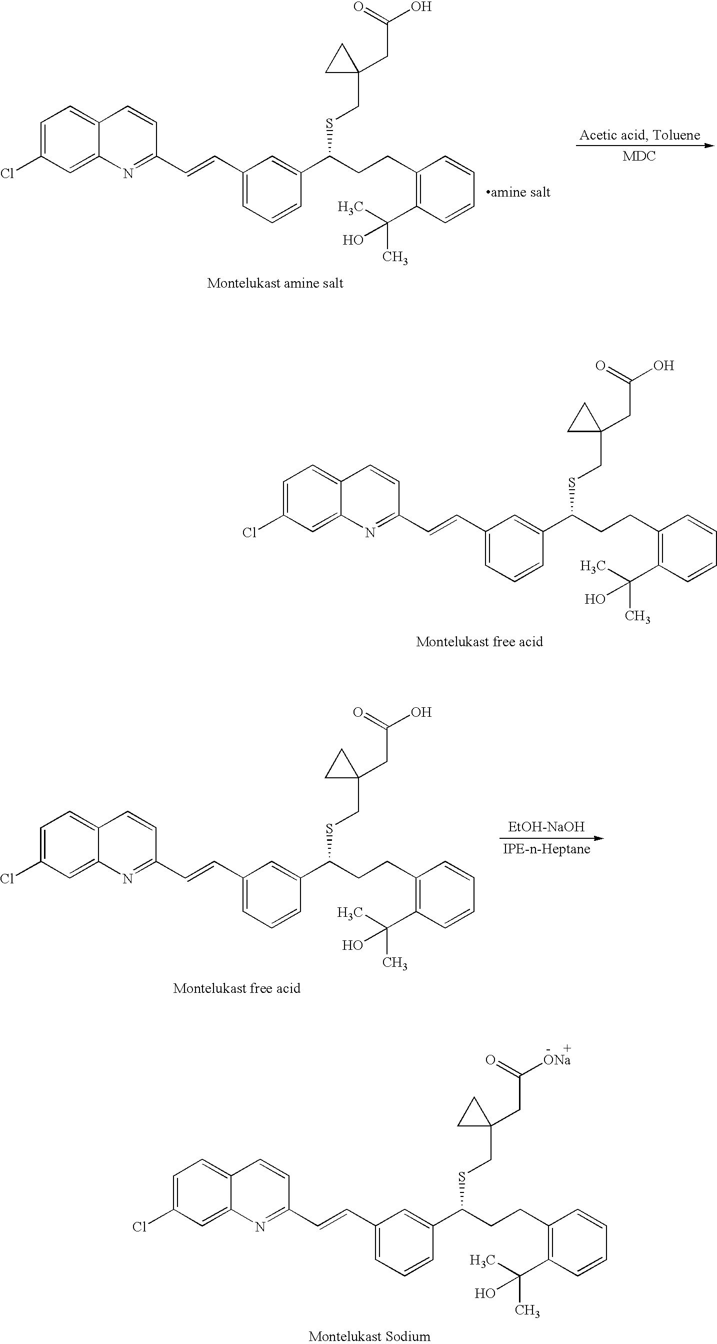

Thus in accordance with the present invention staring material 1-(mercaptomethyl)cyclopropane acetic acid is prepared by the literature reported methods.

Condensation of Methyl-2-[3-[3-(2-(7-chloroquinolin-2-yl)ethenyl]phenyl]-3-chloropropyl]benzoate with 1-(mercaptomethyl)cyclopropane acetic acid is carried out in the presence of alkali carbonates such as Cesium carbonate gives 2-[1-[1(R)-[3-[2-(7-Chloroquinolin-2-yl)ethenyl]phenyl]-3-[2-(methoxy carbonyl)phenyl]propyl sulfanyl methyl]cyclopropane)acetic acid. This reaction is most preferably carried out using dimethyl formamide as the solvent. This reaction can be carried out at a temperature between room temperature and about 100° C., most preferably at 40° C.-60° C. 2-[1-[1(R)-[3-[2-(7-Chloroquinolin-2-yl)ethenyl]phenyl]-3-[2-(methoxy carbonyl)phenyl]propyl sulfanyl methyl]cyclopropane) acetic acid formed above is converted to its organic amine salts such as dicyclohexyl amine, dipropyl amine, α-methyl benzyl amine and cyclohexyl ethylamine salts using respective organic amines in ethyl acetate and isolated the product by adding n-Hexane/n-Heptane. Among the organic amine slats mentioned above dicyclohexylamine slat is the preferable one. The obtained organic amine salt is further purified in toluene and acetonitrile to give pure salt.

2-[1-[1(R)-[3-[2-(7-Chloroquinolin-2-yl)ethenyl]phenyl]-3-[2-(methoxy carbonyl)phenyl]propyl sulfanyl methyl]cyclopropyl)acetic acid amine salt obtained above is neutralized with organic or inorganic acids such as acetic acid, oxalic acid, hydrochloric acid, sulphuric acid preferably acetic acid and the obtained free acid is reacted with methylmagnesium chloride in the presence of anhydrous cerium chloride in an organic solvent preferably tetrahydrofuran or mixture of toluene and THF. Upon completion of the reaction the obtained crude can be converted to its amine salts such as α-methyl benzyl amine salt or cyclohexyl ethylamine salt or Dipropylamine salt. Typically the crude obtained after the reaction completion is treated with (R)-(+)-α-methyl benzyl amine in ethanol and diisoproylether to give 2-[1-[1(R)-[3-[2-(7-Chloroquinolin-2-yl)ethenyl]phenyl]-3-[2-(1-hydroxy-1-methyethyl)phenyl]propyl sulfanyl methyl]cyclopropyl]acetic acid (montelukast) α-methyl benzyl amine salt. α-Methyl benzyl amine salt is further purified in toluene and acetonitrile mixture to give pure montelukast α-methyl benzyl amine salt.

Montelukast α-methyl benzyl amine salt obtained above is neutralized with acid such as hydrochloric acid, acetic acid preferably acetic acid in the presence of methylene dichloride/ethylacetate and water, the organic layer is concentrated and the Montelukast-free acid is isolated in toluene. Similarly concentrated reaction mass can be converted to its sodium salt with out isolating the free acid.

Montelukast free acid is converted to its sodium salt by treating with ethanolic Sodium hydroxide in suitable organic solvent such as Diisopropyl ether, Methyl tert butyl ether, acetone, Ethylacetate, Toluene, Isopropyl acetate, IPA or mixture thereof. Upon completion of the salt formation reaction mass is concentrated and the required sodium salt is isolated in n-Heptane.

The present invention further relates to a process for the preparation of montelukast and its salts comprises of
Treating Methyl-2-[3-[3-(2-(7-chloroquinolin-2-yl)ethenyl]phenyl]-3-chloropropyl]benzoate obtained above with methylmagnesium chloride in the presence of anhydrous cerium Chloride.
Condensing the above obtained compound with 1-(mercaptomethyl)cyclopropane acetic acid in presence of alkali carbonates such as Cesium Carbonate, followed by saltification with organic amine gives montelukast amine salts
Converting the montelukast amine salt to Montelukast Free acid.
Saltification of Montelukast free acid with Sodium Hydroxide affords Montelukast Sodium.

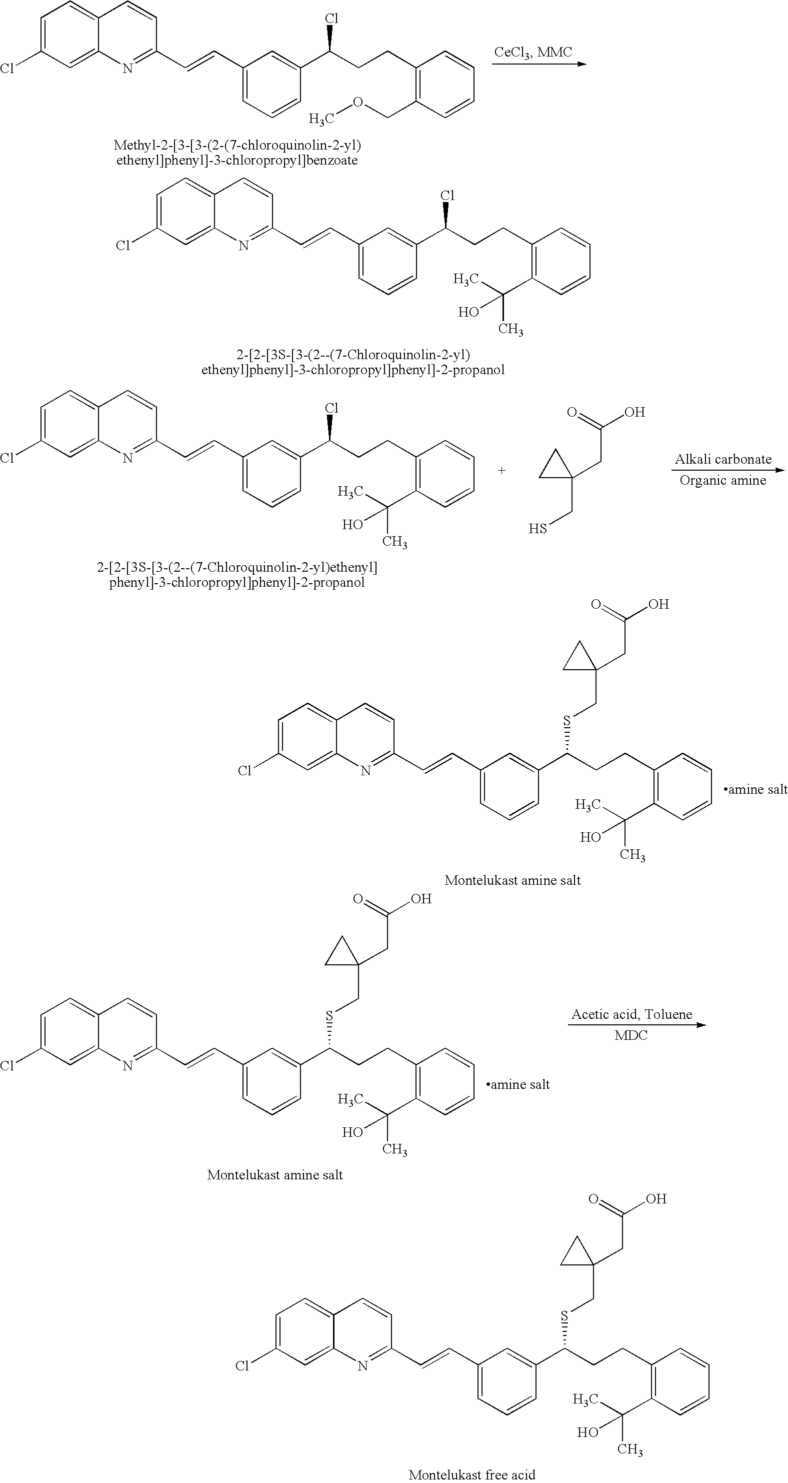

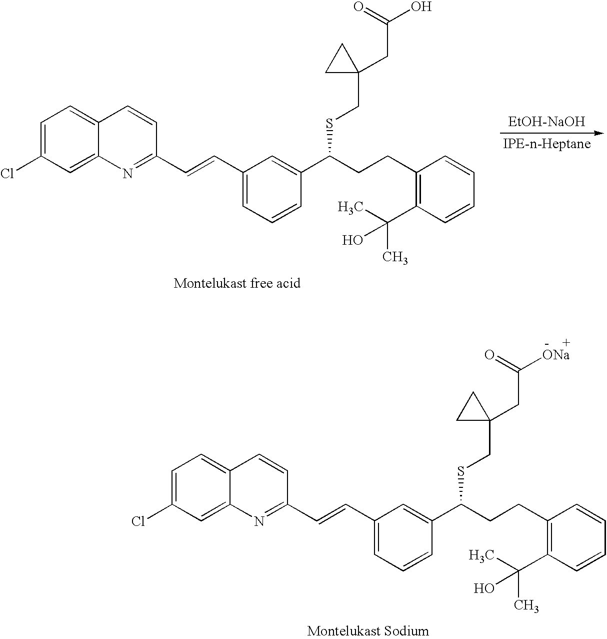

Reaction of Methyl-2-[3-[3-(2-(7-chloroquinolin-2-yl)ethenyl]phenyl]-3-chloro propyl]benzoate with methylmagnesium chloride in the presence of anhydrous cerium chloride in an organic solvent preferably tetrahydrofuran or mixture of toluene and THF yields 2-[2-[3S-[3-[2-(7-Chloroquinolin-2-yl)ethenyl]phenyl]-3-chloropropyl]phenyl]-2-propanol.

2-[2-[3S-[3-[2-(7-Chloroquinolin-2-yl)ethenyl]phenyl]-3-chloropropyl]phenyl]-2-propanol obtained above is reacted with 1-(mercaptomethyl)cyclopropane acetic acid in the presence of alkali carbonates gives 211-[1(R)[3-[2-(7-Chloroquinolin-2-yl)ethenyl]phenyl]-3-[2-(1-hydroxy-1-methyletheyl)phenyl]propyl sulfanyl methyl]cyclopropyl]acetic acid (montelukast). This reaction is most preferably carried out using dimethyl formamide as the solvent. This reaction can be carried out at a temperature between room temperature and about 100° C., most preferably at 40° C.-60° C. 2-[1-[1(R)-[3-[2-(7-Chloroquinolin-2-yl)ethenyl]phenyl]-3-[2-(1-hydroxy-1-methylethyl)phenyl]propyl sulfanyl methyl]cyclopropyl]acetic acid formed above is converted to its organic amine salts such as α-methyl benzyl amine salt or cyclohexyl ethylamine salt or Dipropylamine salt. Typically the crude obtained after the reaction completion is treated with (R)-(+)-α-methyl benzyl amine in ethanol and diisoproylether to give 2-[1-[1(R)-[3-[2-(7-Chloroquinolin 2-yl)ethenyl]phenyl]-3-[2-(1-hydroxy-1-methylethyl)phenyl]propyl sulfanyl methyl]cyclopropyl]acetic acid (montelukast) α-methyl benzyl amine salt. α-Methyl benzyl amine salt is further in toluene and acetonitrile mixture to give pure montelukast (R) (+)-α-methyl benzyl amine salt.

Montelukast (R) (+)-α-methyl benzyl salt obtained above is neutralized with acid such as hydrochloric acid, acetic acid preferably acetic acid in the presence of methylene dichloride/ethyl acetate and water, the organic layer is concentrated and the Montelukast free acid is isolated by addition of toluene, Similarly concentrated reaction mass can also be converted to its sodium salt without isolating the free acid

Montelukast free acid is converted to its sodium salt by treating with ethanolic Sodium hydroxide in suitable organic solvent such as Diisoproylether, Methyl tert butyl ether, acetone, Ethylacetate, Toluene, Isopropylacetate, Isopropanol or mixture thereof. Upon completion of the salt formation reaction mass is concentrated and the required sodium salt is isolated in n-Heptane.

The invention is now illustrated with a few non-limiting examples.

### Example-1

### 2-[1-[1(R)-[3[2-(7-Chloroquinolin-2-yl)ethenyl)phenyl]-3-[2-(methoxy carbonyl)phenyl]propyl sulfanyl methyl]cyclopropyl]acetic acid dicyclohexyl amine salt

1-(Mercaptomethyl)cyclopropane acetic acid (36.8 gms) is dissolved in DMF (600 ml) at room temperature under nitrogen atmosphere. Cesium Carbonate (205.3 gms) is added to the reaction mixture and stirred for 15 minutes. Methyl-2-[3-[3-(2-(7-chloroquinolin-2-yl)ethenyl]phenyl]-3-chloro propyl]benzoate (100 gins) is added to the above mixture in 5-7 equal lots at 30-35° C. Reaction mass temperature is raised to 50-55° C. and maintained for 2-3 hours. Reaction completion is checked by HPLC. Cooled the reaction mixture to 25-35° C. and quenched into a mixture of Ethyl acetate (1000 ml) and 5% Sodium Chloride solution (1000 ml) at temperature below 35° C. over 60 minutes. Stirred for 30 minutes at 25-35° C. and the layers are separated. Aq. Layer is extracted with ethylacetate (1000 ml). Combined both organic layers and washed with 5% Tartaric acid (400 ml), followed by washing twice with 5% Sodium chloride solution (2×1000 ml). Dried the organic layer over anhydrous sodium sulphate (30 gms) and treated with activated charcoal. Reaction mass is filtered over hyflow bed and washed the bed with ethylacetate (100 ml). The clear ethyl acetate layer is transferred into a clean flask and Ethyl acetate is distilled completely under vacuum below 45° C. to get residue.

To the obtained residue Ethyl acetate (600 ml) is charged and the temperature is raised to about 45° C. under nitrogen atmosphere. Cooled the mass to 20-25° C. and Dicyclohexylamine (41.8 gms) is added slowly at 20-25° C. over 30-60 minutes under nitrogen atmosphere. Reaction mass is maintain for 1 hr at 20-25° C. and seeded the reaction mass with the titled compound and stirred for 24 hrs at 20-25° C. under nitrogen atmosphere. n-Hexane (1200 ml) is added to the mass at 20-25° C. over 60 minutes under nitrogen atmosphere, maintained the mass at 20-25° C. for 24 hrs under nitrogen atmosphere. The precipitated salt is filtered and washed the cake with n-hexane (100 ml). Dried the material at 45-50° C. till constant weight Out put: 100 gms

The obtained crude salt (100 gms) is dissolved in a mixture of toluene (450 ml) and Acetonitrile (450 ml) at 50-55° C. under nitrogen atmosphere. Cooled the mass gradually to 30° C. seeded with the pure dicyclohexyl amine salt. Reaction mass is maintained at 28-32° C. for 7-8 hrs under nitrogen atmosphere. The precipitated salt is filtered and washed the cake with acetonitrile (Qty: 50 ml). Dried the product at 45-50° C. till constant weight.

Out put: 50 gms

### Example-2

### 2-[1-[1(R)-[3-[2-(7-Chloroquinolin-2-yl)ethenyl)phenyl]-3-[2-(methoxy carbonyl)phenyl]propyl sulfanyl methyl]cyclopropyl]acetic acid (R) (+)-α-methyl benzyl amine salt

2-[1-[1(R)[3-[2-(7-Chloroquinolin-2-yl)ethenyl)phenyl]-3-[2-(methoxy carbonyl)phenyl]propyl sulfanyl methyl]cyclopropyl]acetic acid can also be isolated as (R) (+)-α-methyl benzyl amine salt is prepared by following the similar procedure as disclosed in example-1.

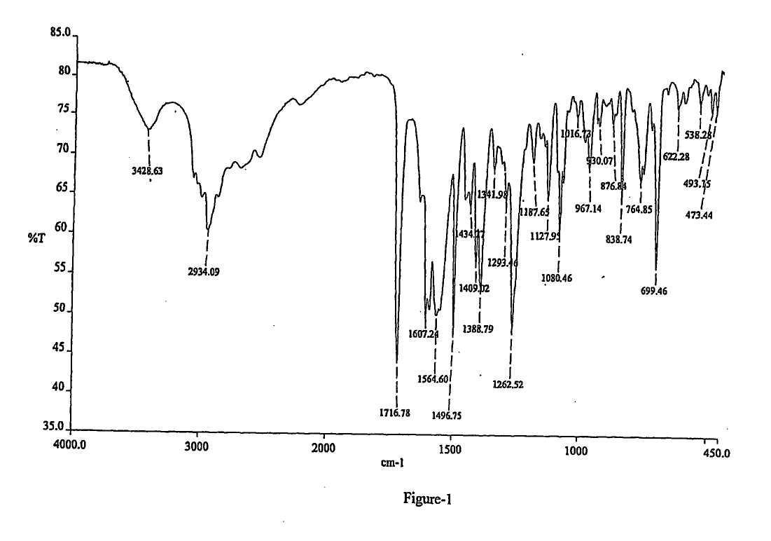

FTIR spectrum is disclosed in FIG. 1

### Example-3

### 2-[1-[1(R)-[3-[2-(7-Chloroquinolin-2-yl)ethenyl)phenyl]-3-[2-(methoxy carbonyl)phenyl]propyl sulfanyl methyl]cyclopropyl]acetic acid cyclohexylethyl amine salt

2-[1-[1(R)-[3-[2-(7-Chloroquinolin-2-yl)ethenyl)phenyl]-3-[2-(methoxy carbonyl)phenyl]propyl sulfanyl methyl]cyclopropyl]acetic acid can also be isolated as cyclohexyl ethyl amine salt is prepared by following the similar procedure as disclosed in example-1.

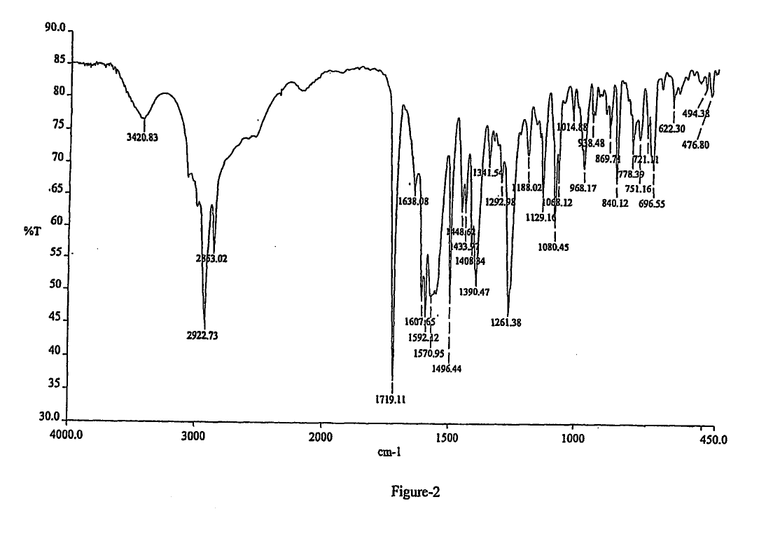

FTIR spectrum is disclosed in FIG. 2

### Example 4

### Preparation of 2-[1-[1-[1(R)-[3-[2-(7-chloroquinolin-2-yl)ethenyl]phenyl]-3-[2-(1-[2-(1-hydroxy-1-methylethyl)phenyl]propyl sulfanyl methyl]cyclopropyl]acetic acid (R) (+)-α-methyl benzyl amine salt

### Part-A

Anhydrous cerium Chloride (38 gms) is added to THF (790 ml.) under Nitrogen atmosphere at room temperature. Temperature is raised to 63° C.-65° C. under Nitrogen and initially distilled off THF (30 ml) for moisture removal by azeotropic distillation. Reaction mixture is maintained for 3 hours at reflux temperature under Nitrogen atmosphere. Cooled the reaction mass to −5° C. under nitrogen. 3.0 M Methyl Magnesium Chloride (380 ml) is added to the reaction mass at −5° C. to 0° C. under Nitrogen atmosphere over 60 minutes.

Reaction mass is maintained for 2 hours at −5° C. to 0° C. under Nitrogen atmosphere.

### Part-B

DCHA salt obtained in example-2 (100 gms) is dissolved in MDC (2000 ml) at room temperature. To the clear solution DM water (1000 ml) is charged and adjust the pH is adjusted 4.0-4.5 with 6% AcOH solution at 20° C.-25° C. Layers are separated and the aqueous layer is extracted with MDC (1000 ml). The combined organic layer is washed with DM water (1000 ml) and dried over Sodium Sulphate (50 gms). The dried organic layer is treated with activated carbon (5 gms) filtered through hyflow bed and washed with MDC (100 ml). MDC is distilled off completely below 40° C. to get yellow residue.

### Part C

The residue obtained in Part-B is dissolved in Toluene (608 ml) at 40° C. and slowly added to Part-A at −5° C. to 0° C. under Nitrogen slowly over 60 minutes. Maintained the reaction mass for 60 minutes at −5° C. to 0° C. under Nitrogen. Reaction completion is checked by TLC and reaction mass is quenched slowly into pre-cooled mixture of 12% acetic acid (1065 ml) and Ethyl Acetate (760 ml) at 5-10° C. over 45 minutes. Stirred the reaction mixture for 10 minutes at 5-10° C. and the temperature is raised to 20° C. and maintain for 30 minutes at 20-25° C. Layers are separated and the aqueous layer is extracted with ethyl acetate (760 ml). Combined organic layers and washed with 10% Sodium Carbonate solution (1220 ml). Followed by 5% NaCl. Solution (2×760 ml) at room temperature. Dried the organic layer with Sodium Sulphate and treated activated carbon at 30° C. Filtered the carbon over hyflow bed and washed with Ethyl Acetate (100 ml). Ethyl acetate is distilled off under vacuum at temperature below 45° C. to get residue.

The obtained residue is dissolved Ethanol (152 ml) at 40-45° C. and cooled the mass to 25° C. (R)-(+)-α-Methyl benzyl amine (20.2 ml) is added slowly at 25-30° C. over 60 minutes under nitrogen. Reaction mass is maintained at 25-30° C. for 1 hour and seeded with (R)-(+)-α-methyl benzyl amine salt. Stirred the mass at 25° C.-30° C. for 8-12 hours under Nitrogen and Di-isopropyl ether (304 ml) is added slowly over 60 minutes at 25-30° C.

Reaction mass is maintained at 25-30° C. for 4 hours under nitrogen and cooled to 10° C. and stirred for 1 hour at 8-10° C. The precipitated slat is filtered and washed with Di-isopopyl ether (50 ml). Wet cake is slurried in Di-isopropyl ether (200 ml) at 25-30° C. for 10-15 minutes, filtered and washed with D-isopropyl ether (50 ml). Dried the material at 45-50° C. till constant weight.

Out put (crude): 70 gms

The above-obtained salt is suspended in a mixture of Toluene (350 ml) and Acetonitrile (1050 ml) at 40-45° C. under nitrogen atmosphere. Reaction mass is maintained for 30 minutes at 40-45° C. and slowly cooled to 30° C. over 30 minutes. Maintained at 28-32° C. for 4 hours and the precipitated salt is filtered and washed with Acetonitrile (60 ml). Dried the product at 45-50° C. till constant weight.

Dry Weight: 57 gms

### Example-5

### Preparation of 2-[1-[1(R)-∂3-[2(7-Chloroquinolin-2-yl)ethenyl]phenyl]-3-[2-(1-hydroxy-1-methylethyl)phenyl]propyl sulfanyl methyl]cyclopropyl]acetic acid cyclohexyl ethyl amine salt

2-[1-[1(R)-[3-[2-(7-Chloroquinolin-2-yl)ethenyl]phenyl]-342-(1-hydroxy-1-methyl ethyl)phenyl]propyl sulfanyl methyl]cyclopropyl]acetic acid can be isolated as cyclohexyl ethylamine salt is prepared by following the procedure as disclosed in example-4

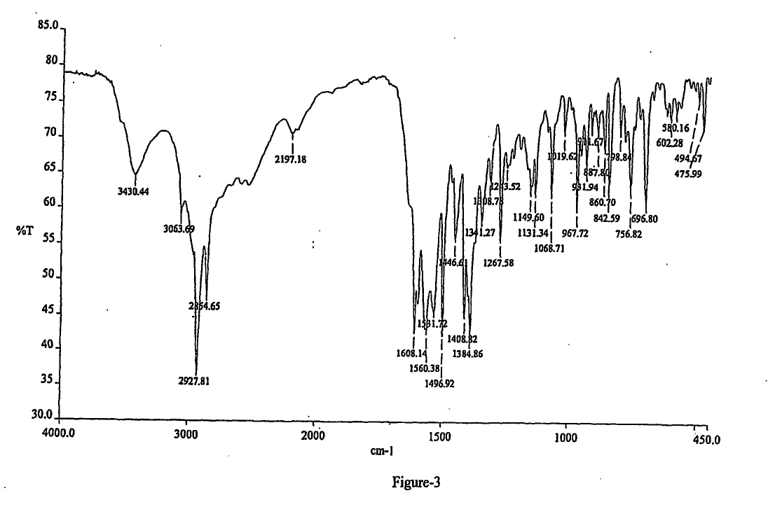

FTIR spectrum is disclosed in FIG. 3

### Example 6

### Preparation of 2-[2-[3S-[3[(2E)-(7-Chloroquinolin-2-yl)ethenyl]phenyl]-3-chloropropyl]phenyl]-2-propanol

### Step-1:

Methyl 2-[(3S)-[3-[(2E)-(7-chloro quinolin-2yl)ethenyl]phenyl]-3-chloropropyl]benzoate (100 g) is suspended in toluene (900 ml) and raised the temperature to 108° C.-110° C., dehydrated by azeotropic distillation and cooled the solution to 20° C.-25° C.

### Step-2:

Anhydrous cerium chloride (50 g) is suspended in THF (1050 ml), raised the temperature of the suspension and distilled off initially 50 ml of THF and maintained the mass at reflux temperature (65° C.) for 3 hours under nitrogen atmosphere. Cooled the reaction mass to −5° C., add 3.0 molar methyl magnesium chloride solution in THF (500 ml) at temperature −5° C.-0° C. over 40 min and maintained for 2 hrs at that temperature. Added step-1 solution to this reaction mass slowly at 0° C.-5° C. and maintained for 2 hrs at temperature of 0° C.-5° C. Transferred the reaction mass into a pre cooled mixture of 12% acetic acid (1400 ml): ethyl acetate (800 ml) at temperature below 20° C. and mixed for 30 min at 18° C.-20° C. Allowed to settle, separated the organic layer, extracted the aqueous layer with ethyl acetate (800 ml), combined organic layer wash successively with 10% sodium carbonate solution (1600 ml), 5%-sodium chloride solution (2×800 ml) and dried the organic layer anhydrous sodium sulphate (15 g). Treated the dried organic layer with activated carbon, distilled off ethyl acetate from the clear solution at temperature below 45° C. under pressure, added ethyl acetate (200 ml) and again distilled off under reduced pressure to get the solid. To the solid added ethyl acetate (100 ml), raised maintained the temperature at 50° C.-55° C. for about 30 min, cooled and maintained at 0° C.-5° C. for 30 min. Filtered the product, washed with pre-cooled ethyl acetate (50 ml) and dried at 45° C.-50° C.

Out put: 65 g

### Example-7

### Preparation of 2-[1-[1(R)-[3-[2(7-Chloroquinolin-2-yl)ethenyl]phenyl]-3-[2-(1-hydroxy-1-methylethyl)phenyl]propyl sulfanyl methyl]cyclopropyl]acetic acid (R) (+)-α-methyl benzyl amine salt

1-(Mercaptomethyl)cyclopropane acetic acid (36.8 gms) is dissolved in DMF (600 ml) at room temperature under nitrogen atmosphere. Cesium Carbonate (205.3 gms) is added to the reaction mixture and stirred for 15 minutes. 2-[2-[3S-[3-[2-(7-Chloroquinolin-2yl)ethenyl]phenyl]-3-chloropropyl]phenyl]-2-propanol (100 gms) is added to the above mixture in 5-7 equal lots at 30-35° C. Reaction mass temperature is raised to 50-55° C. and maintained for 2-3 hours. Reaction completion is checked by HPLC. Cooled the reaction mixture to 25-35° C. and quenched into a mixture of Ethyl acetate (1000 ml) and 5% Sodium chloride solution (1000 ml) at temperature below 35° C. over 60 minutes. Stirred for 30 minutes at 25-35° C. and the layers are separated. Aq.layer is extracted with ethylacetate (1000 ml).

Combined both organic layers and washed with 5% Tartaric acid (400 ml), followed by washing twice with 5% Sodium Chloride Solution (2×1000 ml). Dried the organic layer over anhydrous sodium sulphate (30 gms) and treated with activated charcoal. Reaction mass is filtered over hyflow bed and washed the bed with ethylacetate (100 ml). The clear ethyl acetate layer is transferred into a clean flask and ethyl acetate is distilled completely under vacuum below 45° C. to get residue.

The obtained residue is dissolved ethylacetate (600 ml) at 40-45° C. and cooled the mass to 25° C. (R)-(+)−α-Methyl benzyl amine (33 ml) is added slowly at 25-30° C. over 60 minutes under nitrogen. Reaction mass is maintained at 25-30° C. for 1 hour and seeded with (R)-(+)-α-methyl benzyl amine salt. Stirred the mass at 25-30° C. for 8-12 hours under Nitrogen and n-Heptane (1200 ml) is added slowly over 60 minutes at 25-30° C.

Reaction mass is maintained at 25-30° C. for 12 hours under nitrogen. The precipitated salt is filtered and washed with n-Heptane (50 ml) and dried the material at 45-50° C. till constant weight.

Out put (crude): 80 gms

### Example-8

### Preparation of 2-[1-[1(R)-[3-[2-(7-Chloroquinolin-2yl)ethenyl]phenyl]-3-[2-(1-hydroxy-1-methylethyl)phenyl]propyl sulfanyl methyl]cyclopropyl]acetic acid (S)-cyclohexyl ethylamine salt

Similarly 2-[1-[1(R)[3-[2-(7-Chloroquinolin-2yl)ethenyl]phenyl]-3-[2-(1-hydroxy-1-methylethyl)phenyl]propyl sulfanyl]methyl]cyclopropyl]acetic acid cyclohexyl ethylamine salt is prepared by following the same procedure as disclosed in example-7

### Example-9

### Preparation of 2-[1-[1(R)-[3-[2-(7-Chloroquinolin-2-yl)ethenyl]phenyl]-3-[2-(1-hydroxy-1-methylethyl)phenyl]propyl sulfanyl methyl]cyclopropyl]acetic acid (Montelukast free acid)

Montelukast (R) (+)-α-MBA salt (100 gms) is dissolved MDC (2000 ml) under Nitrogen at 25-30° C. DM Water (1000 ml) is charged and the pH is adjusted to 4.0-4.5 with 6% Acetic Acid at 10-15° C. Temperature is raised to 20° C. and stirred at 20-25° C. for 30 minute. Layers are separated and aqueous layer is extracted with MDC (1000 ml). Combined both organic layers and washed with DM water (1000 ml.) Organic layer is dried over sodium sulphate and treated with activated carbon. Filtered the carbon over hyflow bed and washed with MDC (50 ml). MDC is distilled off under vacuum at temperature below 40° C. to get residue.

Obtained residue is dissolved in Toluene (150 ml) at 45-50° C. Gradually cooled the mass to 25-30° C. under Nitrogen over 60 minutes. Maintained at 25-30° C. for 2 hours under Nitrogen and cooled 10° C. under Nitrogen and stirred for 1 hour at 10-12° C. Precipitated product is filtered and washed with Toluene (50 ml). Dried the product at 45-50° C. till constant weight.

Out Put: 79 Gms

### Example-10

### Preparation of 2-[1-[1(R)-[3-[2-(7-Chloroquinolin-2-yl)ethenyl)phenyl]-3-[2-(1-hydroxy-1-methylethyl)phenyl]propyl sulfanyl methyl)cyclopropyl]acetic acid sodium salt (Montelukast Sodium)

Montelukast free acid (100 gms.) is suspended in Di-isopropyl ether (700 ml) under Nitrogen at 25-30° C. Stirred for 5 minutes and cooled at 8° C. under Nitrogen. 0.486-M Ethanolic NaOH solution (350 ml) is slowly added at 8-12° C. under Nitrogen over 60 minutes. Stirred at 8-12° C. under Nitrogen for 30 minutes and treated with activated carbon at 8-12° C. Filtered the carbon over hyflow bed and washed the bed with Diisopropyl ether (200 ml) at 10-15° C. Filtrate is distilled off completely under vacuum at temperature below 40° C. and apply high vacuum for removing the traces to get white sticky material. n-Heptane (100 ml) is charged to the sticky mass and distilled off solvent traces under vacuum at temperature below 40° C. to get sticky material. Again n-Heptane (2000 ml) is charged at 25-35° C. under Nitrogen. Stirred at 25-35° C. under Nitrogen for 8 hours. Precipitated product is filtered and washed with n-Heptane (200 ml). Dried the product at 50-55° C. under high vacuum for 2 hours 70° C./6 hrs and at 85-95° C. for 3 hours.

Out put: 90 gms

We claim:

1. A process for the preparation of Montelukast or a salt thereof, comprising steps:
i. Condensing Methyl-2-[3-[3-(2-(7-cloroquinolin-2-yl)ethenyl]phenyl]-3-chloro propyl]benzoate with 1-(mercaptomethyl)cyclopropane acetic acid followed by saltification with organic amine
ii. Neutralizing the amine salt obtained above with acetic acid followed by reaction with methylmagnesium chloride in the presence of anhydrous cerium chloride and isolating the product as montelukast amine salt
iii. Converting the montelukast amine salt obtained above to Montelukast Free acid.
iv. Saltification of Montelukast free acid with Sodium hydroxide affords Montelukast Sodium.

2. The process as claimed in claim 1, wherein the condensation reaction of Methyl 2-[(3S)-[3-[2E)-(7-chloroquinolin-2-yl)ethenyl]phenyl]-3-chloropropyl]benzoate with 1-(mercapto methyl)cyclopropane acetic acid is carried out in the presence of alkali carbonates

3. The process as claimed in claim 2, wherein the alkali carbonate is Cesium Carbonate

4. The process as claimed in claim 2, wherein the condensation reaction of Methyl 2-[(3S)-[3-[(2E)-(7-chloroquinolin-2yl)ethenyl)phenyl]-3-chloropropyl]benzoate with 1-(mercaptomethyl)cyclopropane acetic acid is carried out at a temperature between room temperature and about 100° C., most preferable at 40° C.-60° C.

5. The process as claimed in claim 2, wherein the condensed product is isolated as amine salts selecting from dicyclohexylamine salt, cyclohexylethyl amine salt and α-methyl benzyl amine salt.

6. The process as claimed in claim 1, wherein montelukast amine slat is prepared by
i. Contacting the residue obtained after reaction with methyl magnesium chloride with organic amine in ethanol and
ii. Precipitating the amine salt by adding diisopropyl ether

7. The process as claimed in claim 1, wherein the montelukast free acid is isolated by neutralizing the montelukast amine salt

8. The process as claimed in claim 1, wherein the montelukast sodium is prepared by
i. treating the montelukast free acid with sodium hydroxide in a suitable organic solvent
ii. concentrating the reaction mass after salt formation
iii. isolating the montelukast sodium in n-Heptane

9. The process as claimed in claim 9, wherein sodium hydroxide is ethanolic sodium hydroxide

10. The process as claimed in claim 9, wherein suitable organic solvent is selected from Diisopropylether, Methyl tert butyl ether, acetone, Ethylacetate, Toluene, Isopropylacetate, IPA or mixture thereof

11. A process for the preparation of Montelukast or a salt thereof, comprising steps:
i. Reacting Methyl-2-[3-[3-(2-(7-chloroquinolin-2-yl)ethenyl]phenyl]-3-chloropyl]benzoate with methylmagnesium chloride in the presence of Cerium Chloride followed by treatment with organic amine yields amine salts
ii. Neutralizing the organic amine salt as obtained above with organic/inorganic acid followed by condensation with 1-(mercaptomethyl)cyclopropane acetic acid in presence of alkali carbonates such as Cesium Carbonate, followed by saltification with organic amine gives montelukast amine salts
iii. Converting the montelukast amine salt to Montelukast Free acid.
iv. Saltification of Montelukast free acid with Sodium hydroxide affords Montelukast Sodium

12. The process as claimed in claim 12, wherein the alkali carbonate is Cesium Carbonate

13. The process as claimed in claim 12, wherein th Cesium carbonate is used in a molar ratio in a range of 1.5 and 4.5 moles per mole of preparation of 2-[2-[3S-[3-[(2E)-(7-Chloroquinolin-2-yl)ethenyl]phenyl]-3-chloropropyl]phenyl]-2-propanol

14. 2-[1-[1(R)-[3-[2-(7-Chloroquinolin-2yl)ethenyl)phenyl]-3-[2-(methoxycarbonyl)phenyl]propyl sulfanyl methyl]cyclopropyl]acetic acid α-methyl benzyl amine salt characterized by FTIR spectrum as shown in FIG. 1

15. 2-[1-[1(R)-[3-[2-(7-Chloroquinolin-2yl)ethenyl)phenyl]-3-[2-(methoxycarbonyl)phenyl]propyl sulfanyl methyl]cyclopropyl]acetic acid (S)-cyclohexyl ethyl amine salt characterized by FTIR spectrum as shown in FIG. 2.

16. 2-[1-[1(R)-[3-[2-(7-Chloroquinolin-2-yl)ethenyl]phenyl]-3-[2-(1-hydroxy-1-methyl ethyl)phenyl]propyl sulfanyl methyl]cyclopropyl acetic acid (S)-cyclohexyl ethyl amine salt characterized by FTIR spectrum as shown in FIG. 3
